(19) United States
(12) Patent Application Publication
(10) Pub. No.: US 2009/0319191 A1
(43) Pub. Date: Dec. 24, 2009

(54) METHOD FOR DIAGNOSIS OF AN INFECTIOUS DISEASE STAGE AND DETERMINATION OF TREATMENT

(76) Inventors: Ariel L. Rivas, Ithaca, NY (US); Fred W. Quimby, New Durham, NH (US); Michael G. Tokman, Ithaca, NY (US)

Correspondence Address: PASSE' INTELLECTUAL PROPERTY, LLC, 1717 BRASSFIELD RD., RALEIGH, NC, 27614, US

(21) Appl. No.: 12/145,032

(22) Filed: Jun. 24, 2008

(51) Int. Cl. A61B 5/00; G01N 33/50

(52) U.S. Cl. 702/19; 600/300

(57) ABSTRACT

The present invention relates to a means for determining if mammals within a group of mammals is affected by an infection that induces or could induce inflammation, if the mammal is likely to recover or not and if infected whether the mammal should be treated or let to recover untreated. By differentiating infected mammals in a group into different subsets representing differing stages of the progress of the disease the present invention teaches how to prognosticate with continuous data the outcome of a treatment/no treatment decision.

### COPYRIGHT NOTICE

A portion of the disclosure of this patent contains material that is subject to copyright protection. The copyright owner has no objection to the reproduction of the patent document or the patent disclosure by anyone as it appears in the Patent and Trademark Office patent files or records, but otherwise reserves all copyright rights whatsoever.

### BACKGROUND OF THE INVENTION

1. Field of the Invention

The present invention relates to methods and determination of treatment of a mammal or group of mammals having an infectious disease capable of inducing inflammation such as the inflammatory infection of the mammary gland (“mastitis”). Specifically, the present invention relates to dividing a population of mammals into non-infected and multiple infected subsets for the determination which if any subsets should be treated or allowed to run its course untreated.

2. Description of Related Art

Historically, the concept of the process of infectious disease has been associated with the process of inflammation. Both processes have been regarded to be mirror images of each other so measuring the presence of inflammation has been assumed to indicate the presence of infection. By default, absence of inflammation has been taken as evidence of absence of infection. These concepts have had another expression in epidemiology: prevalence (percentage of infected individuals within the total population). The concept of (infectious disease) prevalence is associated with, at least, 2 problems or weaknesses that create errors in treatment choices: (a) it implicitly assumes that all disease-positive cases are identical (each case is assumed to carry the same weight in the calculation of prevalence), and (b) it cannot tell which case is more likely to recover (or, alternatively, to evolve into chronicity, if not death). Accordingly, when prevalence is used, all subjects diagnosed as “disease-positive” are treated and only disease negative subjects are left untreated. However, while treatment decisions become simple using this paradigm, such treatment regime is not consistant with the empirical evidence: two or more “disease-positive” types of infection may occur. Some disease positive cases recover while other disease positive cases result in death. (e.g., the mortality rate is usually much lower than the morbidity rate).

Infectious bovine mastitis is a major health problem of dairy cattle, a herd animal, which results in decreased milk production and decreased milk quality. Staphylococcus aureus is one of the major bacterial pathogens associated with this disease. Identification of bovine mastitis has historically been based on counting all cells present in milk (leukocytes and epithelial cells), also known as somatic cell counts (SCC). Counts greater than 500,000/ml are usually associated with bovine mastitis, which results in reduced milk production and reduced shelf-life of dairy products. The reduction of bovine mastitis prevalence is a major goal of the dairy industry throughout the world. To achieve this goal, most countries ban from the market milk with SCC>500,000 cells/ml, or charge fees for milk deliveries that approach that figure.

In spite of these policies, it is questionable whether measures based on SCC will ever achieve success in decreasing prevalence of bovine mastitis. While high SCC (>1.times.10.sup.6 SSC/ml) is regarded in the industry to be an accurate indicator of bovine mastitis, both mastitic positive and healthy cows can yield an SCC below that figure. The SCC is a generic number that does not take into account the contribution of different inflammatory cell types or leukocytes (i.e., lymphocytes, macrophages and polymorphonuclear cells [or PMN]), nor does it measure cell functions. As a result, neither the number nor the function of each of these three cell types is assessed by the SCC, which prevents accurate diagnosis (determination of present health status) and determination of susceptibility (determination of future health status), as well as evaluation of therapies against mastitis (due to lack of knowledge about their functioning). Furthermore, the SCC does not provide information on the immune status of the individual (i.e., susceptible versus resistant to mastitis). Therefore, there is a need in the dairy industry to develop accurate methods for detection and treatment of mastitis that go beyond the SCC paradigm.

The use of differential cell counts in the diagnosis of mastitis has been proposed for over two decades. Manual milk cytology is the standard technique used to determine leukocyte differential counts. However, the time-consuming nature of, and expertise required by cytologic evaluations of milk leukocytes may be an impediment for current efforts toward improved diagnosis of bovine mastitis. Cytology only allows a relatively low number of cells to be counted per sample. This feature results in inaccurate counts when specimens with low cell concentrations are assessed. Methods have been developed for the diagnosis and susceptibility to bovine mastitis. In U.S. Pat. No. 6,979,550 to Rivas et. al. issued Dec. 27, 2005, there are disclosed methods for separating and determining subsets of herds of cows into no mastitis, early mastitis and late mastitis. These methods however do not go forward to determine when there should or should not be treatment of an infected animal and further do not separate the inflammation/infection paradigm disclosed above. In addition, these methods do not identify disease stages with an objective (statistically defined) procedure.

Accordingly, it would be useful to have a method to determine if an infectious disease is present as well as where it is in the progress of the disease (what specific disease stage it is at) and to determine if treatment of an infection is necessary or not.

### BRIEF SUMMARY OF THE INVENTION

The present invention provides a method for not only determining the stage of infectious disease in more detail than previously known, it also helps a health care practitioner determine if an infected mammal should or should not be treated for the disease and, in addition, it assesses the likelihood of erroneous results of any indicator. By determining the likelihood of a subject to either recover or not recover, treatment plans can be adopted which only treat those subjects unlikely to recover.

In one embodiment the present invention relates to a method for identifying which mammals in an identified group of mammals has one or more diseases selected from the group consisting of infectious and inflammatory diseases and should also receive treatment for the one or more diseases comprising:
a) obtaining leukocyte data on each of the mammals in the group;
b) determining the relationship of major and minor microbial pathogens in the group;
c) dividing the leukocyte data from the group into essentially non-overlapping subsets based on at least one of linearity, microbial profile data and molecular profile data differences;
d) identifying at least 3 subsets representing disease stages wherein each subset exhibits statistical differences in at least one of leukocyte data and microbial data;
e) assessing the relative time involvement of each subset, in which there is no infection, early infection and late infection; and
f) determining if the mammals in each subset should receive treatment.

In yet another embodiment the invention relates to a method of determining the disease state of an individual mammal in a group of mammals comprising:
a) dividing the group into a plurality of biological profile subsets;
b) evaluating the profile subsets for their relevance to disease progress;
c) determining the individual mammal's disease state by assessing which subset the individual mammal is a member of.

Yet another embodiment relates to a method of differentiating the health of a mammal in a group of mammals by evaluating at least two indicators selected from the group comprising the macrophage percentage, the ratio of PMN/lymphocyte percentages, the mononuclear percentage and the PMN/macrophage ratio.

Another embodiment is a method of identifying the progress of an inflammatory infection and the prognosis of the infection in a mammal which is a member of a group of mammals comprising the steps of:
a) determining which members of the group do not have an infection and which have an infection;
b) separating those which have an infection into early inflammatory infection and late inflammatory infection;
c) determining the recovery index for each of the members of the group in late infection group;
d) determining the inflammatory index for each of the members of the late infection group;
e) based on the determined recovery index and inflammatory index of all the members of the late infection group, dividing the group into at least 4 sub-populations comprising an active infection group where major pathogens predominate, an inactive infection group where minor pathogens predominate, a transitional infection group where major pathogens predominate but inflammation is marginal or not observed and an infection without inflammation group where major pathogens predominate,
f) determining for the mammals that have a late inflammatory infection to which sub-population the mammal is a member of.

A method for identifying mammals from a group of infected mammals that do not require treatment comprising evaluating leukocyte data and based on those data not treating mammals which fall into no infection, late transition and late inactive infection groups.

Another embodiment of the invention is a method for identifying which mammals in an identified group of mammals have an infectious disease and should also receive treatment for the disease comprising:
a) obtaining leukocyte data on each of the mammals in the group;
b) obtaining microbial profile data on each of the mammals in the group;
c) dividing the mammals in the group into essentially non-overlapping subsets based on evaluating the leukocyte data based on at least one of linearity and microbial profile;
d) identifying at least 3 subsets which indicate no infection, early infection or late infection; and
e) determining if the mammals in each subset should receive treatment.

Another embodiment is a method for identifying sources of diagnostic errors in the process of testing a group of mammals for an infective disease comprising:
a) producing a leukocyte profile which identifies at least 4 potential diagnostic errors in the testing;
b) producing a microbial profile from the leukocyte profile data of each individual mammal.
c) determining the most likely diagnosis for one or more mammals in the group of mammals based on the microbial profile and the leukocyte profile of the disease stage.

### BRIEF DESCRIPTION OF THE DRAWINGS

FIG. 1 depicts graphs and charts showing the distributions of variables that describe group-level data.

FIG. 2 shows leukocyte profiles in divided subsets.

FIG. 3 shows the distribution of leukocyte and microbial data.

FIG. 4 shows microbial profiles of individual subsets (as disease stages).

FIG. 5 shows leukocyte patterns of disease stages.

FIG. 6 shows leukocyte-microbial differentiation of late mastitis subsets.

FIG. 7 shows molecular confirmation of temporal descriptors.

FIG. 8 shows an assessment of temporal sequence and prognosis of disease state.

FIG. 9 shows a transparent evidence-based assessment of data discrepancies.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, “and an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means “any of the following: A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

By “group of mammals” as used herein is meant a selected limited number of mammals of which information about at least one member of the group is desired. The group could be for example a herd of cows or a group of mammals that shares a measurable condition (e.g., the same spatial location of residence, a given genetic marker). Other non-herd animals such as humans, pets, and the like could be a group under the invention. In one embodiment the group of mammals is a herd of cows.

As used herein an “infectious disease” refers to a microbial infection. Bacteria found in the mammal can be divided into major pathogens and minor pathogens for a given disease. A “major pathogen” is that pathogen(s) which are predominantly associated by one skilled in the art with a particular selected disease. A “minor pathogen” is that pathogen(s) which is a/are minor player(s) in the appearance of a particular disease. Such distinction is well known in the art. For example, in the disease of mastitis in cows, mastitis pathogens are routinely characterized as major and minor. A major pathogen-positive sample for mastitis is designated when Staphylococcus aureus, streptococci, coliforms, Prototheca spp, Arcanobacterium pyogenes, yeast and/or Nocardia spp are isolated at any concentration. Any other organism, chiefly coagulase-negative staphylococci, is considered a minor pathogen in the etiology of bovine mastitis. Major pathogens are the bacteria of concern for causing an infectious disease while minor pathogens are present bacteria but of a lesser or no importance in the mammal overall health.

A “microbial profile” as used herein is the quantitative determination, using continuous data, of the major and minor pathogens that characterize a disease subset and the determination of either the ratio of the percentage of major pathogens to the percentage of the minor pathogens of a given disease stage, or in the alternative, determining the percentage of major pathogens found in a group of samples (e.g., in a disease stage).

As used herein “leukocyte data” refers to the immunological data of each of the mammals in the group. Leukocyte data is the quantification of each leukocyte type (lymphocyte, macrophage, polymorphonuclear cell, i.e. PMN) as either total cell count per milliliter of sample, or relative percentage; as well as the relative ratios involving two or more cell types (e.g., the phagocyte ratio or ratio between the percentage of phagocytes [PMN and macrophages]/percentage of lymphocytes). The term “differential leukocyte count” as used herein means the percentage of lymphocytes, macrophages and polymorphonuclear cells (PMN) among all leukocytes.

The present invention provides methods for the diagnosis of a disease such as mastitis, for identifying multiple subsets representing different stages of infection and, in addition, the ability to evaluate the subsets in such a manner that only those infected subsets that will benefit from treatment are treated and the remaining infected subsets allowed to recover untreated. The method comprises taking a group of mammals such as a herd of cows, and collecting leukocyte data and microbial data on members of the group. The members of the group can be divided then into subsets which not only relate to the stage of disease but also relate to the vector or the progress of the disease and to the need of treatment or the benefit to treatment, if at all.

The algorithms generated by this present invention become a means for therapeutic treatment and evaluation of mammals within the group population in deciding a course or no course of treatment. The following examples and discussion (which are not intended to be restrictive limitations in any way) demonstrate the present invention with a group of cows being evaluated for infectious mastitis.

Microbiology is likely to generate false negatives due to microbial intracellular parasitism. Immunology may generate false negative results due to microbial intracellular parasitism, immune depletion, and/or microbial modulation of the immune response. By integrating microbiological with immunological data, analyzing the data on the basis of partitioned testing (an approach that does not assume health/disease is composed of only two [dichotomous] outcomes), and applying well-established statistical procedures (which address microbial profiles and inflammatory stages), cases are differentiated and analyzed with emphasis on estimating the evolution over time of these leukocyte-microbial profiles.

While leukocyte profiles indicative of inflammation are associated with microbial profiles that, first, indicate infection and later, recovery (findings that may be applied as a new prognostic system), lack of inflammation does not always indicate absence of infection: a leukocyte subset may indicate infection but not inflammation (i.e., “I w/o I”). If so, the classical paradigm is not accurate in its dichotomic assumption: absence of inflammation does not necessarily indicate absence of infection.

Data available in the public domain are used to demonstrate this method. A dataset (data collected from a group) is the first unit of analysis. Such dataset contains, if available, the date the samples were collected, the spatial location (e.g., the latitude and longitude of a farm), the mammal identifier, and the identifier of the organ where the sample is collected (e.g., the mammary gland quarter of a cow).

In addition, the counts/ml and percentage of lymphocytes, macrophages, and PMN of the tested organ are recorded, as well as the microbiologic results (e.g., microbial isolation or lack of isolation [of a specific microbial species and/or microbial subspecies or strain, if indicated]). Other indicators may be included, although they are not required, such as the total cell count (not discriminating for each leukocyte type).

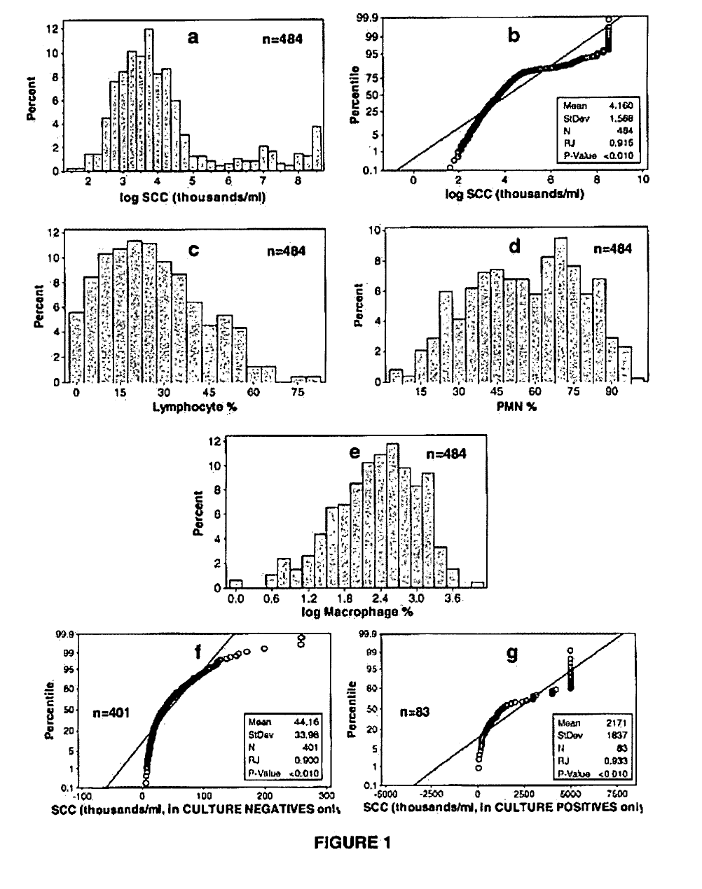

Then, the data distribution is determined (FIG. 1). Typically, the distribution of a given indicator (e.g., the lymphocyte percentage) is non-linear when the whole dataset (all the lymphocyte percentages of all the mammals being tested) is analyzed (FIGS. 1a-g).

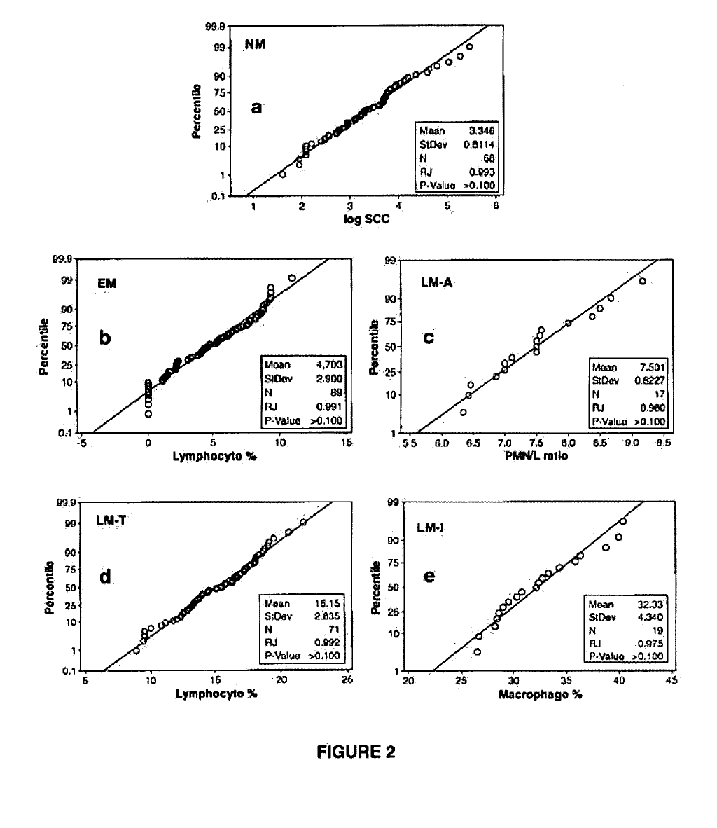

Subsequently, an open-ended procedure is conducted with the purpose of dividing the dataset in as many subsets as produced by the process such that, each one is linearly distributed (FIGS. 2a-e). This procedure is finalized when at least three criteria are met: 1) at least 5 subsets are generated (displaying linearity as determined by established statistical methods), 2) not more than one additional subset is created by default (a subset defined by the remaining, linearly distributed ones), and 3) each so created subset differs statistically from the remaining ones in, at least, two indicators (e.g., the range of the lymphocyte % values and the range of the PMN % values of one subset should differ from the ranges of those indicators in, at least, two other disease subsets).

This procedure can be implemented by any means (e.g., a computer software, a manual process). However, the end goal is the same: the 3 criteria indicated above.

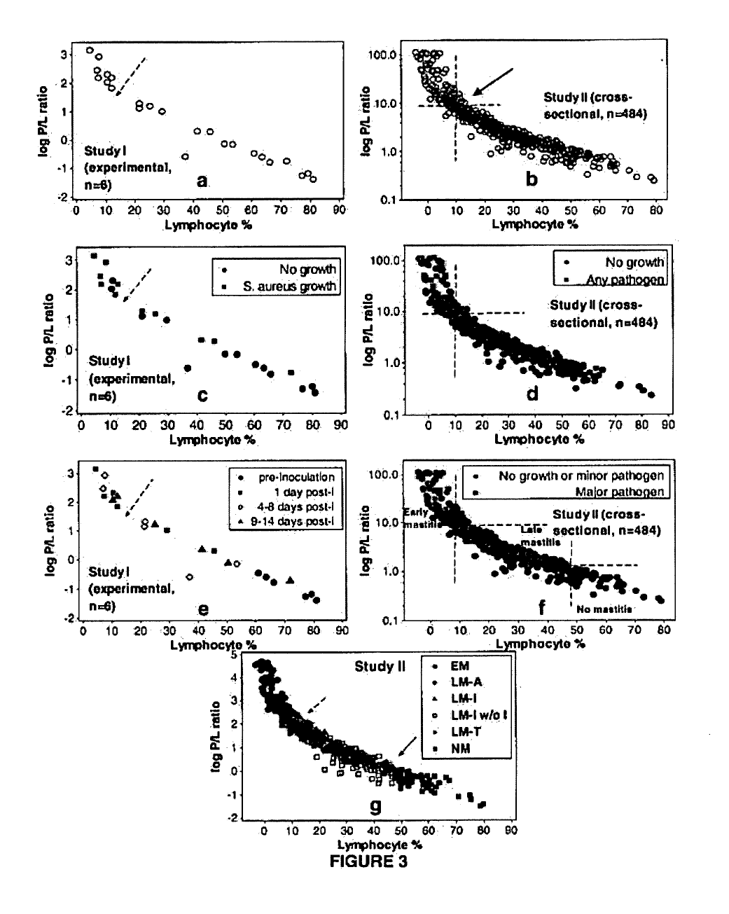

Once at least 5 subsets displaying linearity have been identified, a bi-dimensional plot of 2 leukocyte indicators (e.g., the distribution of the lymphocyte percentage data vs. the distribution of the [log-transformed] phagocyte/lymphocyte ratio) is produced with the purpose of identifying at least one major inflexion point (FIG. 3a).

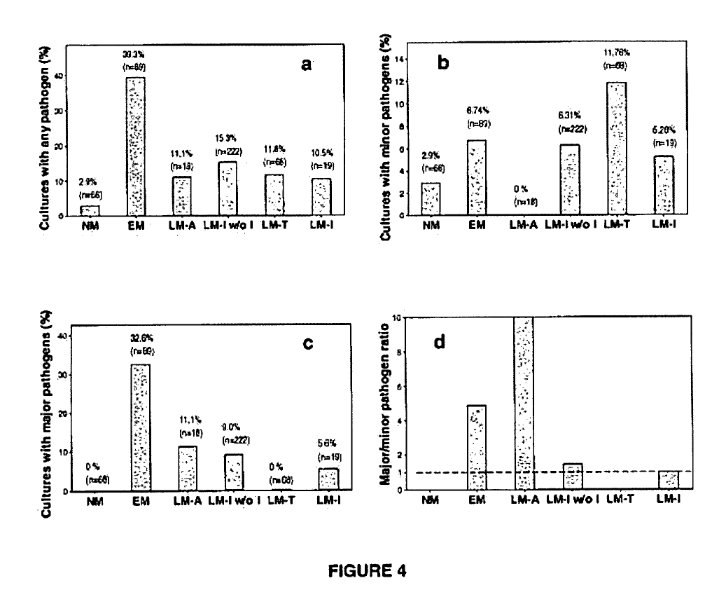

Then, the microbial profiles corresponding to each disease subset (disease stage) are determined. Both the percentage of major pathogens and the major/minor pathogen ratio are calculated (FIGS. 4a-d).

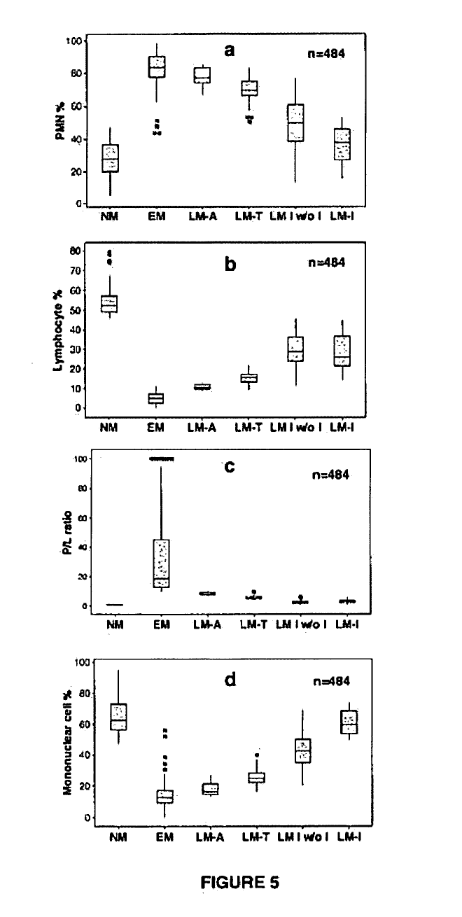

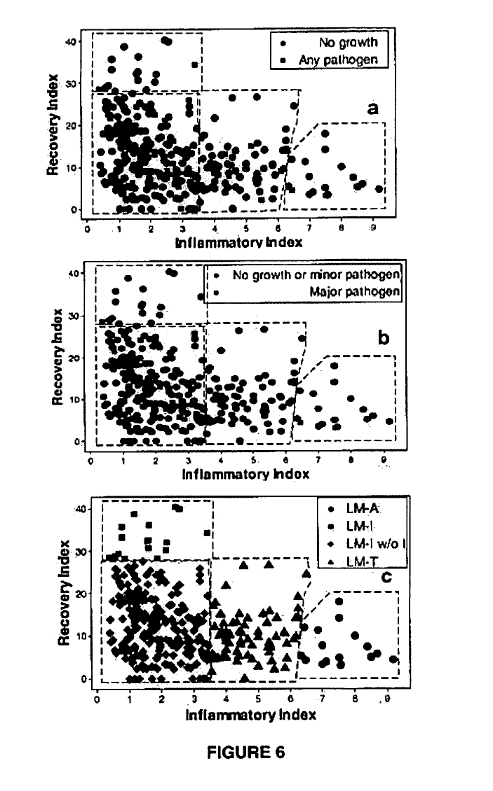

After confirming that at least 2 disease stages can be differentiated statistically by, at least, 2 leukocyte indicators (FIGS. 5a-d), a pair of (individual or composite) leukocyte indicators is used to confirm that the “late” inflammation group of samples can be subdivided into at least 4 subsets (FIGS. 6a-c). Based on the temporal assignment of each disease subset (FIG. 3g and FIGS. 7 and 8), prognosis can be produced.

For samples falling within the disease subsets named “late inflammation (“mastitis”) and inactive” (LM-I) a “recovery” prognosis is reported. For samples falling within the disease subset named “infection without inflammation” (LM-I w/o I), a “reserved” (unlikely to recover) prognosis is reported.

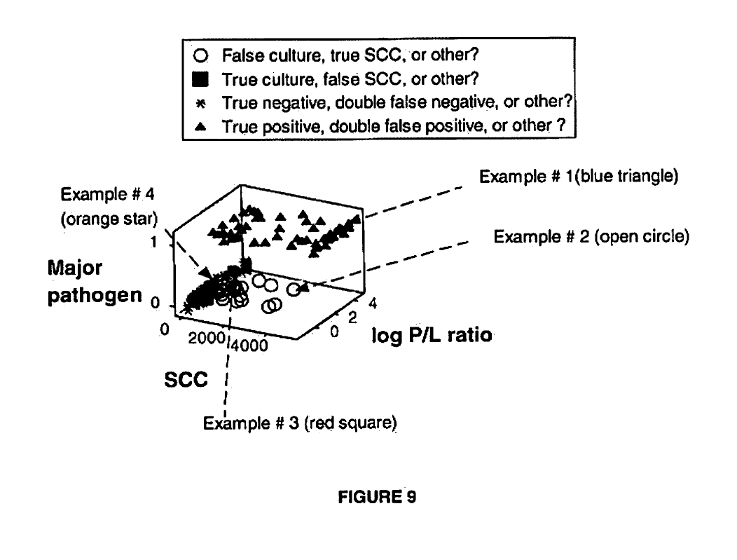

By using any set of at least 4 non-overlapping rules, questions are attached to the report of each individual sample to prompt diagnosticians for the likelihood of erroneous results. For instance, if the total cell count [“SCC”] is higher than a certain value [a pre-determined threshold, e.g. 200,000 cells/ml] and no bacterium was isolated in culture, a “true culture, false SCC, or other?” question is created (FIG. 9). The microbial profiles that characterize each disease subset are also reported.

### DETAILED DESCRIPTION OF THE DRAWINGS

FIG. 1 describes the group-level (population level) data distributions of mammary gland cell-related variables. Four variables are explored: 1) the somatic cell count/ml (SCC [thousands]/ml, a, b), 2) the lymphocyte% (c), 3) the polymorphonuclear cell (PMN) % (d), 4) the macrophage % (e). Regardless of whether the data are not-transformed (c, d) or log-transformed (a, e), lack of linearity is observed when whole (non-fragmented) group-level datasets are plotted (a). A statistical test for data linearity (Ryan-Joiner or RJ) indicates that the data are not linearly distributed (P value <0.05, b). Lack of linearity is not due to bacteriological status: when the same dataset (n=484) is divided into culture-negative (bacteria-negative, n=401) and culture-positive (bacteria-positive, n=83) subsets, both subsets displayed non-linearity (f, g).

FIG. 2 describes the result of the data fragmentation and merging process. The same data described in FIG. 1 are subject to an undeterminate number of combinations until the original dataset is divided into subsets that reveal linearity. Using an algorithm that facilitates the division of the original dataset into subsets that differ from each other in at least 2 indicators (e.g., the lymphocyte and the PMN percentages) while observations falling within the same subset display similar values for the same leukocyte indicators, the process of data partitioning (and merging) continues until most (if not all) observations are grouped into (as many) linearly distributed subsets (as the analysis of a specific population may require). This process creates at least 5 subsets displaying such structure (a-e). The insert within each plot indicates: 1) the mean value and the standard deviation of the indicated indicator for the indicated subset (e.g., a mean of 4.7% lymphocytes for the EM subset [b]), 2) the size of the subset (n=89, for the EM subset [b]), and 3) the P value of the Ryan Joiner (RJ) statistical test for linearity which, if >0.05, indicates linearity cannot be rejected. Individual data points not included in these subsets constitute an additional group, defined by default. The acronyms used to identify each subset are provisional identifiers, proposed according to data ranges consistent with the literature: “no mastitis” (NM), “early mastitis” (EM), late mastitis, active (LM-A), “late mastitis, transitional stage” (LM-T), and “late mastitis, inactive” stage (LM-I). Later assessments (FIGS. 3-8) explore whether such temporal descriptors are adequate.

FIG. 3 identifies each observation of a population on the basis of 4 descriptors: 1) microbial, 2) leukocyte, 3) disease stage, and 4) relative time. Public records are used to demonstrate the implementation of this process (a-g). In the left column, the results of an experimental and longitudinal study are shown (“study I”, n=6, 4 temporal measurements, a, c, e). The chronological time when observations were made (in relation to the initiation of the experiment [“days post-i”]) is shown in e. In the right column, the collective results of a dataset (“study II”, n=484 samples) measured at a single time point are shown. When microbial data are added (isolation in culture of S. aureus—the only pathogen tested in the experimental study—[c], or any microbial pathogen [d]), no bacterial isolation (no infection) is observed in the lower right quadrant, while a higher percentage of bacterial isolation (infection) is observed in the left upper quadrant (above the arrow [c] or at the intersection between two perpendicular lines [d]). When either the exact chronological time is determined (e) or only major pathogens are considered (f), it is noticed that observations seen in the upper left quadrant correspond to the earliest infection (early mastitis), which are characterized by a higher percentage of major pathogens (including S aureus). The point where major pathogens are no more observed is indicated as a second intersection between perpendicular lines (f). Hence, early mastitis observations (characterized by a higher percentage of major pathogens) are located above the arrow (to the left and above the major inflection point). Late mastitis (blue points in e) are defined by default (f): they are those located between the major inflexion point and the point where the last major pathogen is observed (between intersected lines). Therefore, [1] the major inflection point (characteristic of each population) separates the area of early mastitis (to its left and above) from the area of late mastitis (below and to its right), and [2] the end of the late mastitis area is indicated by the point where the last major pathogen is observed. The area of late mastitis and that of no mastitis is also determined by plotting (over imposing) the values of the different disease stages determined by the linearity-oriented process described in FIG. 2(g). By using both the linearity-oriented data fragmentation process and the major inflection point (shown here) as an additional confirmation point, disease stages are characterized objectively (a data-driven, population-specific process is produced).

FIG. 4 describes the microbial profiles of individual disease stages with continuous data. The microbial profile of 6 disease stages (the 5 linearly-defined subsets [shown in FIG. 2] and an additional subset here identified as LM-I w/o I, which is created by default [all the observations not included in the remaining subsets) is shown (a-d). Using the same dataset analyzed before (“study II”, n=484) and expressed as the ratio between the percentage of samples with major pathogens/the percentage of samples with minor pathogens (d), clear differences are observed between the group including EM, LM-A, and LM-I w/o I (all with a major/minor pathogen ratio above 1, i.e., true infection) and the group including NM, LM-T and LM-I (all showing a major/minor pathogen ratio≦1, no infection). Similar patterns are seen when the data are expressed as percentage of major pathogens (c).

FIG. 5 describes the leukocyte profiles of individual disease stages with continuous data. The use of numerous leukocyte indicators differentiates the 6 data subsets (disease stages, a-d). While some indicators do not differentiate all stages (e.g., the Lymphocyte % does not differentiate LM-I w/o I from LM-I, b), those disease stages are differentiated by the Mononuclear cell % (d). Vice versa, the Mononuclear cell % does not differentiate EM from LM-A, but these disease stages are differentiated by the Lymphocyte %. The collective use of multiple leukocyte indicators provides numerous opportunities to differentiate disease stages.

FIG. 6 describes the late infection (mastitis) subsets with 3 leukocyte and 2 microbial indicators. Several composite indices can be used to characterize both the extension of the inflammatory response (“inflammatory index”) and its resolution (“recovery index”). In this example, the ratio of PMN/lymphocyte percentages is used as “inflammatory index”, while the percentage of macrophages (limited to values found within the range of late mastitis observations) is used as “recovery index.” Other combinations of composite indices can produce alternative “inflammatory” and “recovery” indices (e.g., the log PMN/Macrophage ratio and the Lymphocyte %, respectively). The example (based on “study II” data) shows that the percentage of bacterial isolations does not vary at random: there are more bacteria isolated in the lower, left quadrant (“LM-I w/o I”), and most of them are major pathogens. In contrast, LM-T does not show any major pathogen, and LM-I shows only one. LM-A differs markedly from LM-I: (a) the 2 bacterial isolations of LM-A are major pathogens, (b) the “recovery index” of LM-A shows values only half of those of LM-I, and (c) the “inflammatory index” of LM-A is about 3 times higher than that of LM-I. Leukocyte-microbial plots confirm the non-overlapping nature of the 6 disease stages generated by the linearity-oriented, data fragmentation and merging process.

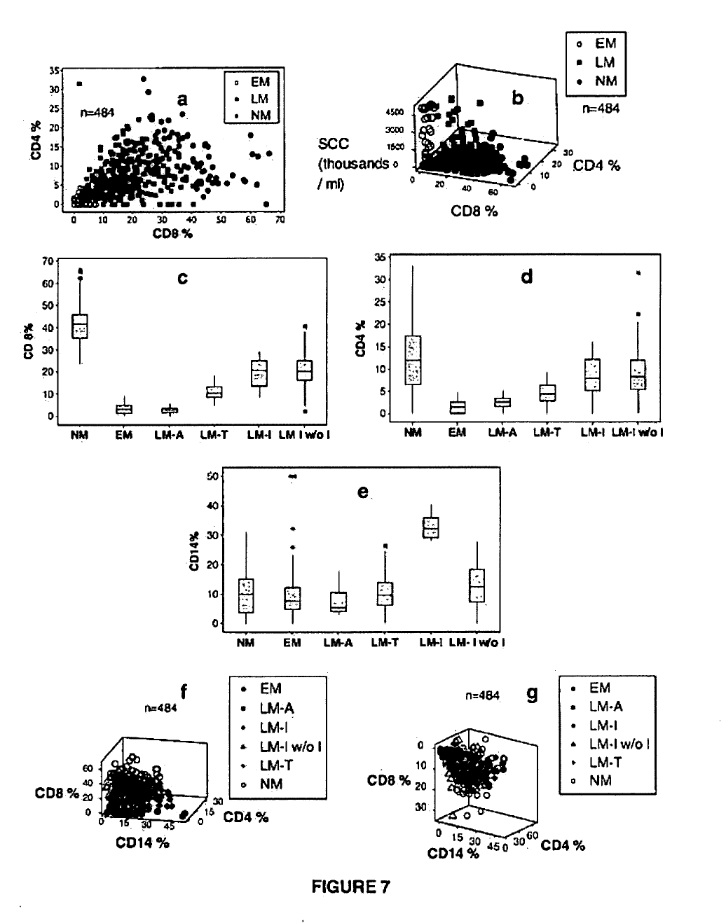

FIG. 7 confirms the temporal descriptors assigned to each disease stage, using leukocyte molecular data. Because leukocyte molecular markers (CD4, CD8, CD14) are known to vary in expression at different inflammatory phases (e.g., the percentage of milk lymphocytes expressing CD8 is greater than that of CD4+ milk lymphocytes in non-infected mammals, while CD4 predominate over CD8 in early mastitis, but not in late mastitis), then, the relative expression of these markers (together with that of macrophage CD14) may be used as proxy indicators of time (not chronological time, but “inflammatory” time). It is shown that CD4 and CD8 patterns are not overlapping (they differentiate NM, EM, and LM, a). In contrast, the SCC does not distinguish these 3 major subsets (e.g., SCC<1,500,000 cells/ml can be seen both in NM, EM, and LM, b). These markers differentiate some but not all disease stages (c-e). For instance, neither CD4 nor CD8 differentiates LM-I from LM-I w/o I. However, CD14 differentiates LM-I from LM-I w/o I (e). Their collective use differentiates all 6 disease stages, indicating non-overlapping ranges (f, g), (g shows a different perspective of the plot shown in f). These sub-cellular leukocyte (molecular) data confirm (with 484 observations) the temporal descriptors (no, early, late) estimated before based on a small longitudinal study (“study I”) and, in addition, these data justify the temporal descriptors assigned to “late” subsets (assigned on the basis of data partitioning until all observations are included into linearly-distributed subsets and not more than one additional disease stage is created by default, FIG. 2), demonstrating that they are not overlapping.

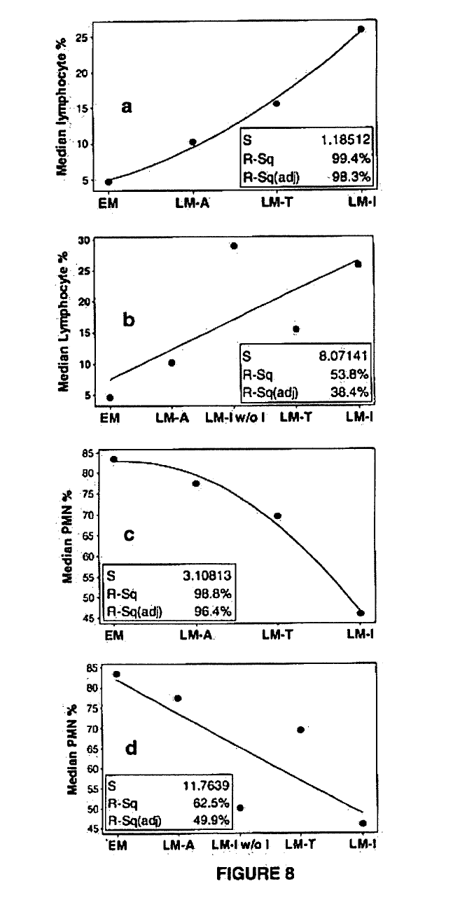

FIG. 8 describes an analysis that supports the prognosis attributed to two of the 4 late disease stages. The temporal order of infectious disease stages (excluding “no mastitis”) is assessed (a-d). The question of interest is to determine which subset precedes or follows which. To that end, each disease stage was assigned a relative time unit (e.g., “time unit I”, “time unit II”, time unit III”, “time unit IV”, and “time unit V”). First, only 4 temporal units were considered and only 4 disease stages were analyzed. All 24 possible combinations were analyzed. For example, in the first 6 tests, EM was assigned “time unit I” (see the list below) while the remaining 3 temporal units were occupied by the remaining disease stages.

| | Time units (I to IV) | | | | | | | |
|---|---|---|---|---|---|---|---|---|
| | I | II | III | IV | I | II | III | IV |
| Disease stage | EM | LM-A | LM-T | LM-I | LM-T | EM | LM-A | LM-I |
| | EM | LM-A | LM-I | LM-T | LM-T | EM | LM-I | LM-A |
| | EM | LM-T | LM-I | LM-A | LM-T | LM-A | EM | LM-I |
| | EM | LM-T | LM-A | LM-I | LM-T | LM-A | LM-I | EM |
| | EM | LM-I | LM-A | LM-T | LM-T | LM-I | EM | LM-A |
| | EM | LM-I | LM-T | LM-A | LM-T | LM-I | LM-A | EM |
| | LM-A | EM | LM-T | LM-I | LM-I | EM | LM-A | LM-T |
| | LM-A | EM | LM-I | LM-T | LM-I | EM | LM-T | LM-A |
| | LM-A | LM-T | EM | LM-I | LM-I | LM-A | EM | LM-T |
| | LM-A | LM-T | LM-I | EM | LM-I | LM-A | LM-T | EM |
| | LM-A | LM-I | EM | LM-T | LM-I | LM-T | EM | LM-A |
| | LM-A | LM-I | LM-T | EM | LM-I | LM-T | LM-A | EM |

This analysis used the time unit of each disease stage as the predictor (horizontal axis) and several leukocyte indicators as the response indicator (vertical axis in the plots). Two indicators (a, c) yielded the highest regression coefficient (R² adj≧96.4%) when EM was time unit I (earliest), LM-A was time unit II, LM-T was time unit III, and LM-I was time unit IV (latest). However, once 5 temporal units were assessed and LM-I w/o I was included in the analysis (b, d), the regression coefficient decreased markedly (R² adj≦49.9%) These findings suggest that: a) EM precedes LM-A, b) LM-A precedes LM-T, and c) LM-T precedes LM-I. Findings are also consistent with the hypothesis that these disease stages are components of the same (a single) process. It also shows that their temporal order is associated with their leukocyte values. However, LM-I w/o I does not seem to fit within that sequence/process. Because LM-I is the last phase (and next to NM, as shown in FIG. 3g), observations falling within the LM-I stage are regarded to be under recovery. Because LM-I w/o I does not fit within this temporal sequence (there is no evidence that it precedes either LM-I or NM), LM-I w/o I is not assumed to be under recovery.

FIG. 9 describes examples of the report produced by this method, which includes a transparent, evidence-based assessment of data discrepancies.

This method also assesses its own chances for erroneous results. For instance, if a sample shows SCC above a certain threshold (e.g. 200,000 cells/ml) but no major pathogen is isolated, a question is asked: “is it a true culture result, a false SCC result, or other explanation is plausible?” Here, 4 such questions were asked to each of 484 observations. By plotting the questions generated by each result and producing a table where that question is shown as an additional column (see table below), diagnosticians are alerted about a possible cause of concern. By showing the microbial profile associated with the observed leukocyte profile, diagnosticians are provided with the data that can respond to the question of interest. The most defensible diagnosis is also provided.

Example #1 asks: “Is it a true positive, a double false positive, or other result (is plausible)?” The data do not show discrepancies. The report shows a high SCC (5,000,000 cells.ml), a major pathogen is isolated, PMN % is 85%, and no lymphocytes are observed (see table below). The microbial profile associated with these leukocyte profiles includes (a) 32.58% of major pathogens, and (b) a major/minor pathogen ratio of 4.83. The most defensible diagnosis is EM.

Example #2 is prompted by the question: “False culture, true SCC, or other?” It shows what seems to be a false negative culture (see table below). It is a sample that produced a high SCC (3,000,000 cells/ml) but a negative culture. In addition, that sample also produced a high PMN percentage (86.7%), a low lymphocyte percent (5.3%), and a high phagocyte/lymphocyte ratio (17.0). These leukocyte profiles are associated with a 32.58% of major pathogen+cultures, and a major/minor pathogen ratio of 4.83. These 5 pieces of information are complementary with the SCC, supporting the hypothesis of an EM result. Had the traditional, “gold standard”-based paradigm been used (which uses microbiological results as the reference), this sample would have been diagnosed as “disease negative” when, in spite of an erroneous microbiological result, it is most likely a “disease positive” result.

Example #3 shows no bacterial isolation and 547,000 SCC/ml. While the percentage of PMN is lower (and that of lymphocytes is higher) than displayed by the previous examples, such pattern still differs from that of a truly non-infected animal. A diagnosis of LM-I w/o I is the most defensible.

Example #4 shows a low SCC (20,000 cells/ml) and no bacterial isolation. This pattern is linked to the question: “True negative, double false negative, or other?” Such pattern could suggest a lack of infection. However, the PMN/M and the P/L ratio are higher than in a NM case. The most likely diagnosis is neither “true negative” nor “double false negative”: this pattern can be observed in a LM-T case. This example demonstrates the practical use of this system. Had the conventional (dychotomous) paradigm been applied, this sample could have been misdiagnosed: it could have been diagnosed “no inflammation” when, in fact, the leukocyte profile suggests it belongs to a “late inflammation” stage.

TABLE 1

| | Culture, | | | | | | | | | | | | |
|---|---|---|---|---|---|---|---|---|---|---|---|---|---|
| | 0 = no or | | | | | | | | | | | | |
| | minor, | SCC | | | | | | | | | Associated | Associated | |
| | 1 = major | (1 × 10³ | L | M | | PMN | PMN/M | PMN/L | | P/L | Major | M/m path. | Likely |
| Ex^{a} | pathogen | cells/ml) | %^{a} | %^{a} | Mono %^{a} | %^{a} | ratio | ratio | Phago %^{a} | ratio | path. % | ratio | Dx^{a} |
| #1^{b} | 1 | 5000 | 0 | 11.2 | 11.2 | 85.7 | 7.6 | 99.9* | 86.9 | 99.9* | 32.58 | 4.83 | EM |
| #2^{c} | 0 | 3000 | 5.3 | 4.0 | 9.3 | 86.7 | 21.7 | 16.2 | 90.7 | 17.0 | 32.58 | 4.83 | EM |
| #3^{d} | 0 | 547 | 13.8 | 14.9 | 28.7 | 71.3 | 4.8 | 5.2 | 86.2 | 6.3 | 9.0 | 1.43 | LM-I |
| | | | | | | | | | | | | | w/o I |
| #4^{e} | 0 | 20 | 17.2 | 5.2 | 22.4 | 67.2 | 13.0 | 3.9 | 72.4 | 4.2 | 0.0 | 0.0 | LM-T |

^{a}Ex = example, L = lymphocyte, M = macrophage, Mono = mononuclear cell, PMN = polymorphonuclear cell, Phago = phagocyte (PMN and macrophage), Dx = diagnosis, 99.9 = infinity. Leukocytes do not always add up to a 100% since they were identified by flow cytometry.
^{b}“True positive, double false positive, or other?”
^{c}“False culture, true SCC, or other?”
^{d}“True culture, false SCC, or other?”
^{e}“True negative, double false negative, or other?”

The claims which follow are not intended to be limited by the preceding examples. Variations in methods, diseases, or mammalian species are possible and considered within the scope of the claims of the present invention.

What is claimed is:

1. A method for identifying which mammals in an identified group of mammals has one or more diseases selected from the group consisting of infectious and inflammatory diseases and should also receive treatment for the one or more diseases comprising:
a) obtaining leukocyte data on each of the mammals in the group;
b) determining the relationship of major and minor microbial pathogens in the group;
c) dividing the leukocyte data from the group into essentially non-overlapping subsets based on evaluating the leukocyte data based on at least one of linearity and microbial profile;
d) identifying at least 3 subsets representing disease stages wherein each subset exhibits statistical differences in at least one of leukocyte data and microbial data;
e) assessing the relative time involvement of each subset, in which there is no infection, early infection and late infection; and
f) determining if the mammals in each subset should receive treatment.

2. A method according to claim 1 wherein the group of mammals is a group of herd mammals.

3. A method according to claim 1 wherein the group of mammals is a group of non-herd mammals.

4. A method according to claim 1 wherein the disease is an intra-mammary gland infection capable of inducing inflammation.

5. A method according to claim 1 which further comprises obtaining the leukocyte data with microbial status.

6. A method according to claim 1 wherein there are at least two subsets indicating at least one of late infection and late inflammation.

7. A method of determining the disease state of an individual mammal in a group of mammals comprising:
a) dividing the group into a plurality of biological profile subsets;
b) evaluating the profile subsets for their relevance to disease prognosis; and
c) determining the individual mammal's disease state by assessing which subset the individual mammal is a member of.

8. A method of differentiating the health of a mammal in a group of mammals by evaluating at least two indicators selected from the group comprising the macrophage percentage, the ratio of PMN/lymphocyte percentages, the mononuclear percentage and the PMN/macrophage ratio.

9. A method of identifying the progress of an inflammatory infection and the prognosis of the progress of the disease in a mammal which is a member of a group of mammals comprising the steps of:
a) determining which members of the group do not have an infection and which have an infection;
b) separating those which have an infection into early inflammatory infection and late inflammatory;
c) determining the recovery index for each of the members of the late infection group;
d) determining the inflammatory index for each of the members of the late infection group;
e) based on the determined recovery index and inflammatory index of all the members of the late infection group, dividing the group into at least 4 sub-populations comprising an active infection group where major pathogens predominate, an inactive infection group where minor pathogens predominate, a transitional infection group where major pathogens predominate but inflammation is marginal or not observed and an infection without inflammation group where major pathogen predominate; and
f) determining for the mammals that have a late infection to which sub-population the mammal is a member of.

10. A method according to claim 9 wherein the inflammatory infection is mastitis in a lactating mammal.

11. A method according to claim 10 wherein the determination is used to decide if the mammal should be treated for mastitis or to let the mastitis run its course without treatment.

12. A method according to claim 10 wherein the determination of which sub-population the mammal is in is used to decide if the mammal should receive treatment for mastitis.

13. A method according to claim 10 wherein mammals determined to be in late mastitis are grouped into more than 4 sub-populations.

14. A method according to claim 10 wherein within the late mastitis mammals only the active infection group and infection without inflammation group are treated for mastitis.

15. A method for identifying which mammals in an identified group of mammals have an infectious disease and should also receive treatment for the disease comprising:
a) obtaining leukocyte data on each of the mammals in the group;
b) obtaining microbial profile data on each of the mammals in the group;
c) dividing the mammals in the group into essentially non-overlapping subsets based on evaluating the leukocyte data based on at least one of linearity and microbial profile;
d) identifying at least 3 subsets which indicate no infection, early infection or late infection; and
e) determining if the mammals in each subset should receive treatment.

16. A method for identifying mammals from a group of infected mammals that do not require treatment comprising evaluating leukocyte data and based on those data not treating mammals which fall into no infection, late transition and late inactive infection groups.

17. A method for identifying sources of diagnostic errors in the process of testing a group of mammals for an infective inflammatory disease comprising:
a) producing a leukocyte profile which identifies at least 4 potential diagnostic errors in the testing;
b) producing a microbial profile from the leukocyte profile data of each individual mammal; and
c) determining the most likely diagnosis for one or more mammals in the group of mammals based on the microbial profile and the leukocyte profile of the disease stage.
